(19) United States
(12) Patent Application Publication
(10) Pub. No.: US 2026/0369372 A1
(43) Pub. Date: Nov. 19, 2026

(54) ANALYSIS DEVICE, PROGRAM, ANALYSIS SYSTEM, AND ANALYSIS METHOD

(71) Applicant: KONICA MINOLTA, INC., Tokyo (JP)

(72) Inventors: Tomoaki TAKEMURA, Hachioji-shi, Tokyo (JP); Shinichi HORITA, Osaka-shi, Osaka (JP); Seiji NOMURA, Hino-shi, Tokyo (JP); Hiroshi TERAO, Hino-shi, Tokyo (JP)

(21) Appl. No.: 19/133,930

(22) PCT Filed: Nov. 14, 2023
(86) PCT No.: PCT/JP2023/040940

§ 371 Date: May 29, 2025

(30) Foreign Application Priority Data
Nov. 29, 2022 (JP) 2022-190198

(51) Int. Cl. G16H 15/00; G06F 40/20; G06F 40/40

(52) U.S. Cl. CPC G16H 15/00; G06F 40/20; G06F 40/40

(57) ABSTRACT

Disclosed is an analysis device including a hardware processor that performs natural language analysis on an interpretation report to determine information relating to an important finding and outputs a determination result.

### TECHNICAL FIELD

The present invention relates to an analysis device, a program, an analysis system, and an analysis method.

### BACKGROUND ART

Conventionally, in the medical field, it is a common practice for a radiologist to create an image diagnosis report (interpretation report) for a medical image captured by a modality. The clinician makes a diagnosis while checking the interpretation report created by the radiologist.

The information on whether or not the interpretation report is an important interpretation report including an important finding is important information for confirming the presence or absence of an important finding. Therefore, this information is important for a confirming doctor (a clinician, a side who views the interpretation report) who is a checker of the interpretation report. Here, the important finding is an important disease name or finding.

Patent Document 1 describes that when a creator (radiologist) selects a range of a word or phrase that the creator wants a checker (clinician) to check with a pointer 102 using a mouse 18 and selects a level of importance degree of the range, identification display corresponding to the selected level is provided in the selected range.

In addition, Patent Document 2 describes that a predetermined character string included in a character string list 331 of a storage 33 is extracted from a first interpretation report, and a second interpretation report 90 in which checkboxes C1 to C15 are provided at positions corresponding to the extracted predetermined character string in the first interpretation report is created.

### CITATION LIST

### Patent Literature

Patent Document 1: Japanese Patent No. 5215454

Patent Document 2: Japanese Unexamined Patent Publication No. 2019-204189

### SUMMARY OF INVENTION

### Technical Problem

In Patent Document 1, it is necessary for the radiologist to select a word or phrase of an important finding and register a level, but it is not mandatory for the radiologist to perform these registration operations, and it is not always necessary for the radiologist to perform registration. Therefore, if the radiologist does not register an important finding or forgets to register an important finding, the checker (clinician) may miss the important finding.

In addition, in Patent Document 1, there is also a possibility that the checker (clinician) misses an important finding because the phrase and level of the important finding are not registered due to the insufficient skill of the radiologist. For example, in Patent Document 1, it is an object to prevent a confirmer (clinician) from making a mistake in response. However, for example, selection of an important finding and its level may be different depending on the proficiency of the creator (radiologist), and the important finding may be overlooked.

Furthermore, the creator (radiologist) may miss registration of an important finding. That is, whether or not the checker (clinician) misses an important finding depends on the creator (radiologist).

Furthermore, in Patent Document 2, if the first interpretation report includes a predetermined character string stored in the character string list 331, all of the character string is extracted. For example, in a case where the character string of “nodule shadow” is included in the character string list 331, the character string of “nodule shadow” is extracted even in a case of the interpretation report having content that “nodule shadow is not recognized”. Therefore, a situation occurs in which a confirmer (clinician) has to perform unnecessary confirmation. Since a large number of unimportant character strings are extracted in this way, for example, an important finding such as “Observe nodule shadows” may be missed.

The present invention has been made in consideration of the above-described problem in the conventional technology and has as its object to prevent a person who checks an interpretation report from missing an important finding.

### Solution to Problem

In order to solve the above problem, the analysis device of the present invention includes
a determination section that performs natural language analysis on an interpretation report to determine information relating to an important finding; and
an output section that outputs a determination result by the determination section.

In addition, the program of the present invention causes a computer to achieve:
determining function of performing natural language analysis on an interpretation report and determining information relating to an important finding; and
outputting function of outputting a determination result by the determining function.

Furthermore, the analysis system according to the present invention includes
a determination section that performs natural language analysis on an interpretation report to determine information relating to an important finding; and
an output section that outputs a determination result by the determination section.

Furthermore, the analysis method of the present invention includes
determining of performing natural language analysis on an interpretation report to determine information relating to an important finding; and
outputting of outputting a determination result in the determining.

### Advantageous Effects of Invention

According to the present invention, it is possible to prevent a checker of an interpretation report from missing an important finding.

### BRIEF DESCRIPTION OF DRAWINGS

FIG. 1 This is a configuration diagram of the report system.

FIG. 2 This is a block diagram illustrating the functional configuration of a report server.

FIG. 3 This is a diagram showing an example of the dictionary management screen.

FIG. 4 This is a block diagram illustrating a functional configuration of an image interpretation terminal and a reference terminal.

FIG. 5 This is a flowchart illustrating a flow of interpretation report creation processing executed by the controller of FIG. 2.

FIG. 6 This is a diagram showing an example of the report list screen.

FIG. 7 This is a diagram showing an example of the report input screen.

FIG. 8 This is a diagram for explaining determination by the determination section.

FIG. 9 This is a view illustrating an example of the first confirmation screen.

FIG. 10 This is a view illustrating an example of the second confirmation screen.

FIG. 11 This is a diagram showing another example of the second confirmation screen.

FIG. 12 This is a diagram showing an example of the report reference screen.

### DESCRIPTION OF EMBODIMENTS

Hereinafter, embodiments of an analysis device, a program, an analysis system, and an analysis method of the present invention will be described. However, the scope of the invention is not limited to the illustrated examples.

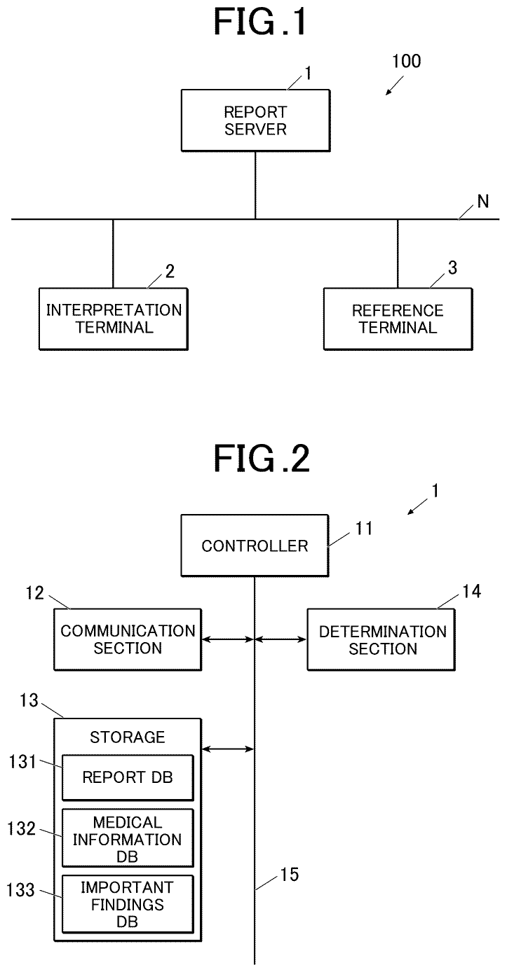

FIG. 1 is a diagram illustrating an example of a configuration of a report system 100 as an embodiment of an analysis system of the present invention. The report system 100 is a system for creating, analyzing, and managing a interpretation report on a medical image.

As illustrated in FIG. 1, the report system 100 includes a report server 1, an image interpretation terminal 2, and a reference terminal 3. The devices constituting the report system 100 are connected via a network N such as a LAN (Local Area Network) so as to be capable of data communication. In addition, each apparatus constituting the report system 100 conforms to the HL7 (Health Level Seven) or DICOM (Digital Image and Communications in Medicine) standard, and communication between the apparatuses is performed in accordance with HL7 or DICOM.

Note that the number of the image interpretation terminals 2 and the reference terminals 3 is not particularly limited. In addition, a hospital information system (HIS), a radiology information system (RIS), a modality, a picture archiving and communication system (PACS), and the like (not illustrated) are connected to the communication network N.

The report server 1 is a server that provides a function for a radiologist to create an interpretation report in the interpretation terminal 2 and for a clinician (confirmation doctor) to refer to the interpretation report in the reference terminal 3. The report server 1 has a built-in function as an analysis device of the present invention.

FIG. 2 is a block diagram illustrating the functional configuration of the report server 1.

As shown in FIG. 2, the report server 1 includes a controller 11, a communication section 12, a storage 13, a determination section 14, and the like, and each unit is connected by a bus 15.

The controller 11 includes a central processing unit (CPU), a read only memory (ROM), a random access memory (RAM), and the like. The controller 11 comprehensively controls processing operation of each section of the report server 1. Specifically, the CPU of the controller 11 reads various processing programs stored in the ROM, develops the programs in the RAM, and performs various kinds of processing, such as the interpretation report creation processing described later, in cooperation with the programs. The controller 11 functions as an output section, a generation section, and an acquisition section.

The communication section 12 is constituted by a network interface or the like, and transmits and receives data to and from an external device connected via the communication network N.

The storage 13 includes a hard disk drive (HDD), a nonvolatile memory, and the like. The storage 13 stores various programs and data.

In the present embodiment, the storage 13 is provided with a report database (DB) 131. The report DB 131 includes a report management table that stores management information on the interpretation report and a report storage area that stores the interpretation report. The report management table includes, for example, items such as inspection information, patient information, identification information of an interpretation report, a determination result of the interpretation report by the determination section 14, and a confirmed flag, and stores information of these items in association with each interpretation report as one record. The inspection information includes information such as an inspection ID, an inspection date, a modality, and a medical department. The patient information includes information such as a patient ID, a patient name, gender, age, and date of birth. The identification information of the interpretation report is a report ID or the like. The determination result of the interpretation report by the determination section 14 includes information on the presence or absence of an important finding, the type of important finding, the level of important finding, the department, and the like. Note that in the present embodiment, the report management table stores, as a determination result of the presence or absence of an important finding, an important finding flag ON if the determination result indicates the presence of an important finding and an important finding flag OFF if the determination result indicates the absence of an important finding. The report storage area stores an interpretation report in association with a report ID.

Here, when the reservation of the inspection is registered, an RIS (not illustrated) notifies the report server 1 of the inspection information and the patient information of the reserved inspection. In the report server 1, the controller 11 registers the inspection information and the patient information on the reserved inspection notified by the RIS as an inspection for which an interpretation report is to be created, in the report management table of the report DB 131.

Furthermore, the storage 13 is provided with a medical information DB 132 (medical information dictionary) and an important finding DB 133 (important finding dictionary). The medical information DB 132 and the important finding DB 133 are databases used for the determination section 14 to determine information relating to important findings from the finding text of the interpretation report.

Here, the important finding is a diagnostically important disease name or finding. In the present embodiment, the important finding is a disease name or a finding whose disease name information or finding information is stored in the important finding DB 133. The information relating to the important finding includes, for example, the presence or absence of the important finding, the type of the important finding, the level of the important finding, and the department handling the important finding. The types of important finding include types of urgency of important finding, such as urgent and normal. The level of an important finding includes the degree of importance of the important finding.

The medical information DB 132 is a database that stores medical information such as a disease name, a site (anatomical name), and a finding. The medical information DB 132 includes, for example, a disease name DB, an anatomical name DB, and a finding DB. The disease name DB stores disease name information (e.g., lung carcinoma, emphysema, carcinoma, and---). The anatomic name DB stores anatomic name information (for example, A1, A10, A1 Ward, A2, A2 Ward, and---) of a site. The finding DB stores finding information (tumor, nodule, shadow, infiltration, mass, and---).

The important finding database 133 is a database that stores, as important finding information, diagnostically important disease name information or important finding information among disease name information or finding information. The important finding information includes, for example, important disease name information or important finding information (lung cancer, nodule, and the like). Furthermore, the important finding information is stored with an application range (department), a type (urgent/normal), and a level (A, B, C, or the like) in an associated manner.

Note that the medical information DB 132 and the important finding DB 133 may be provided for each clinical department.

The user can add, modify, and delete information stored in the medical information DB 132 and the important finding DB 133 from the interpretation terminal 2.

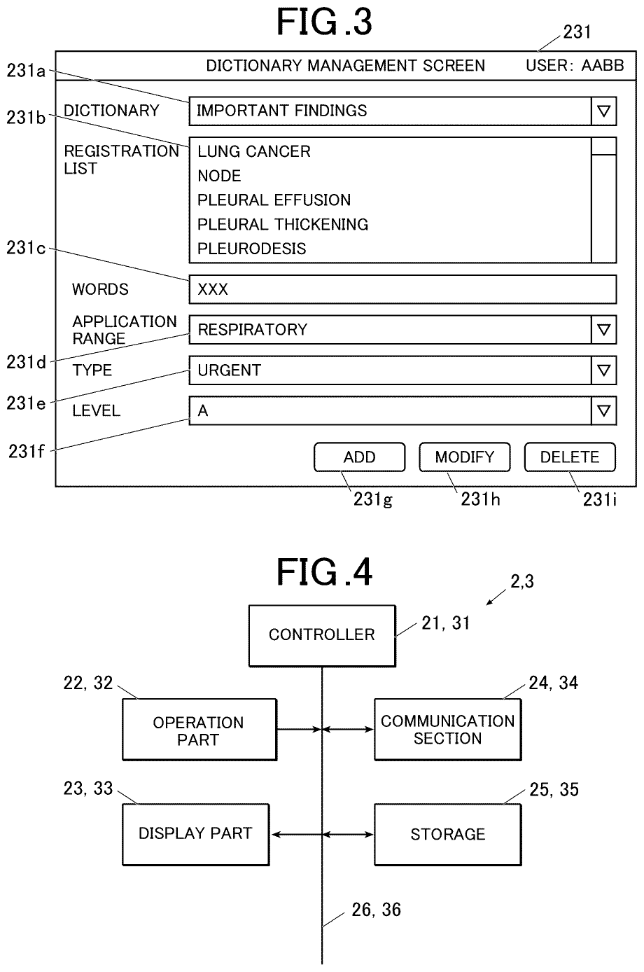

FIG. 3 is a diagram showing an example of a dictionary management screen 231 for adding, correcting, and deleting information to and from the medical information DB 132 and the important findings DB 133. As illustrated in FIG. 3, the dictionary management screen 231 is provided with a dictionary selection field 231a, a word selection field 231b, a word input field 231c, an application range selection field 231d, a type selection field 231e, a level selection field 231f, an add button 231g, a modify button 231h, and a delete button 231i.

The dictionary selection field 231a is a field for selecting a dictionary (DB) to be added, corrected, or deleted as a target. The word selection field 231b is a field for selecting a target word to be corrected or deleted from a list of information (words) registered in the dictionary (DB) selected in the dictionary selection field 231a. The word input field 231c is a field for inputting a target word to be added. The application range selection field 231d is a field for selecting a medical care department to which the change of the dictionary is applied. The type selection field 231e is a field for selecting whether a word (disease name, finding, or the like) to be added, corrected, or deleted is urgent or normal (not urgent). The level selection field 231f is a field for selecting the level of a word (disease name, finding, or the like) to be added, corrected, or deleted as a target. The add button 231g is a button for giving an instruction to additionally register a word input in the word input field 231c in a DB selected in the dictionary selection field 231a. The correction button 231h is a button for instructing to correct the word selected in the word selection field 231b. The button 231i is a button for giving an instruction to delete the word selected in the word selection field 231b from the DB selected in the dictionary selection field 231a.

When a request to display the dictionary management screen 231 is received from the image interpretation terminal 2, the controller 11 outputs (transmits) screen information on the dictionary management screen 231 to the image interpretation terminal 2 via the communication section 12. When instruction information of addition, correction, or deletion input on the dictionary management screen 231 is transmitted from the interpretation terminal 2, the controller 11 receives the instruction information, and adds, corrects, or deletes the selected word in the selected DB based on the received instruction information.

The determination section 14 performs natural language analysis on the interpretation report to determine information relating to important findings.

Natural language analysis (natural language processing) refers to analyzing, by a machine, a language that humans use on a daily basis to analyze the content of the language. Predetermined character strings are registered in the database, and searching for a character string from the database and extracting the character string are not included in the natural language analysis. The natural language analysis is processed in the flow of, for example, morphological analysis, syntactic analysis, semantic analysis, and contextual analysis.

In the present embodiment, the determination section 14 is implemented by software processing in cooperation with the program stored in the storage 13 and the CPU of the controller 11. The natural language analysis performed by the determination section 14 includes at least one of syntactic analysis, semantic analysis, and contextual analysis. Details of the analysis by the determination section 14 will be described later.

The interpretation terminal 2 is a computer device such as a personal computer (PC) used by a radiologist. The radiologist interprets the medical image, creates an interpretation report, and the like at the image interpretation terminal 2.

FIG. 4 is a block diagram illustrating the functional configuration of the image interpretation terminal 2.

As shown in FIG. 4, the image interpretation terminal 2 includes a controller 21, an operation part 22, a display part 23, a communication section 24, a storage 25, and the like, and each unit is connected by a bus 26.

The controller 21 includes a CPU, a ROM, a RAM, and the like, and integrally controls the processing operation of each unit of the interpretation terminal 2. Specifically, the CPU reads various processing programs stored in the ROM, develops the read programs in the RAM, and carries out various processes in cooperation with the program.

The operation part 22 includes a keyboard including cursor keys, character/number input keys, various function keys, and the like, and a pointing device such as a mouse. The operation part 22 outputs an operation signal input by a key operation on the keyboard and a mouse operation to the controller 21.

The display part 23 includes a monitor such as a liquid crystal display (LCD). The display part 23 displays various screens according to an instruction of a display signal input from the controller 21.

The communication section 24 is configured by a network interface and the like, and transmits and receives data to and from an external device connected via a communication network N.

The storage 25 is formed with an HDD, a nonvolatile semiconductor memory, or the like, and stores various types of data.

The reference terminal 3 is a computer device such as a computer used by a clinician (confirmation doctor). The clinician confirms the interpretation report or the like in the reference terminal 3.

FIG. 4 is a block diagram illustrating a functional configuration of the reference terminal 3.

As illustrated in FIG. 4, the reference terminal 3 is configured to include a controller 31, an operation part 32, a display part 33, a communication section 34, a storage 35, and the like, and each unit is connected by a bus 36. Each unit of the reference terminal 3 is the same as each unit of the image interpretation terminal 2, and therefore, description is omitted.

Next, operation performed in the report system 100 will be described.

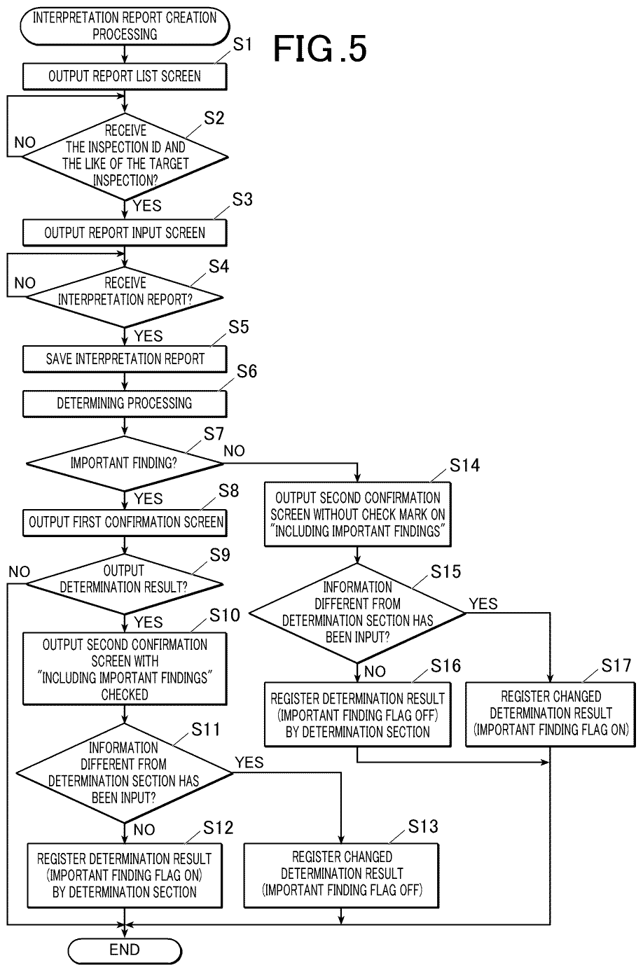

FIG. 5 is a flowchart illustrating the flow of interpretation report creation processing performed in the report server 1 in response to access from the image interpretation terminal 2. The interpretation report creation processing is executed by the cooperation of the CPU of the controller 11 of the report server 1 and the program stored in the ROM.

First, the controller 11 generates screen information of the report list screen 232 based on the inspection information and the patient information stored in the report management table of the report DB 131, and outputs (transmits) the screen information to the image interpretation terminal 2 by the communication section 12 (step S1).

The image interpretation terminal 2 displays the report list screen 232 on the display part 23 based on the screen information received from the report server 1.

For example, the controller 11 generates a screen to be displayed on the interpretation terminal 2 or the reference terminal 3 as a Web screen, and outputs the screen information to the interpretation terminal 2 or the reference terminal 3. The image interpretation terminal 2 or the reference terminal 3 displays, by a web browser, a screen based on the screen information output from the report server 1.

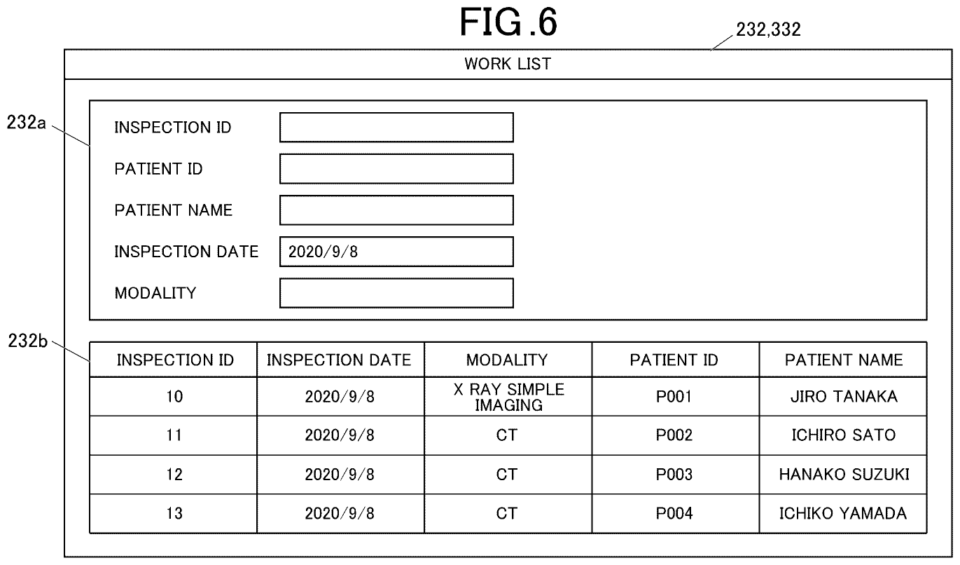

FIG. 6 is a view illustrating an example of a report list screen 232. As shown in FIG. 6, the report list screen 232 is provided with a narrowing-down condition input field 232a and a list display field 232b. The narrowing-down condition input field 232a is a field for inputting a narrowing-down condition for the inspection information and the patient information to be displayed in the list display field 232b. The display field 232b is a field for displaying a list of the inspection information and the patient information of the report creation targets matching the conditions input in the narrowing-down condition input field 232a.

When the inspection information and the patient information of the inspection for which the report is to be generated are selected from the inspection information and the patient information displayed in the display field 232b, the controller 21 of the interpretation terminal 2 transmits the inspection IDs and the like of the selected inspections to the report server 1 via the communication section 24.

Upon receiving the inspection IDs and the like of the selected inspections for which reports are to be generated from the image interpretation terminal 2 (step S2; YES), the controller 11 generates screen information on the report input screen 233 for inputting the interpretation report on the selected inspections and outputs the screen information to the image interpretation terminal 2 via the communication section 12 (step S3).

The image interpretation terminal 2 displays the report input screen 233 on the display part 23 based on the screen information received from the report server 1.

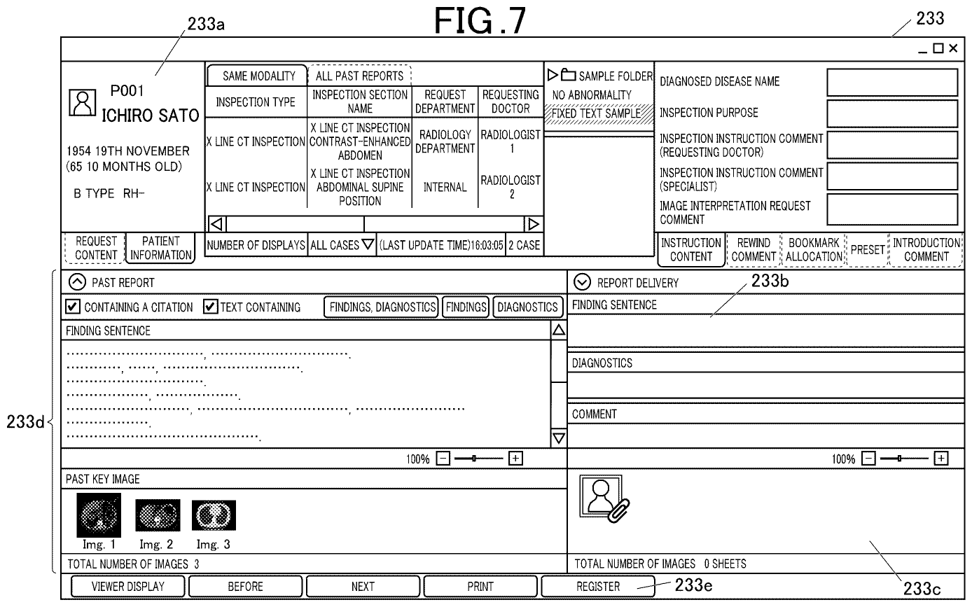

FIG. 7 is a view illustrating an example of the report input screen 233. As illustrated in FIG. 7, the report input screen 233 is provided with an information display field 233a, a finding sentence input field 233b, a medical image addition field 233c, a past report display field 233d, and a registration button 233e.

The information display field 233a is a field for displaying patient information and inspection information of an inspection for which a report is to be created. The finding sentence input field 233b is a field in which the radiologist inputs a finding sentence, for example, in text. The finding sentence input here is unstructured data that is not structured. The medical image attachment field 233c is a field to which a thumbnail image of a medical image obtained in an inspection is attached. The past report display field 233d is a field in which an interpretation report for a past examination of the same patient and a thumbnail image attached to the interpretation report are displayed. The registration button 233e is a button for giving an instruction to register the input interpretation report in the report DB 131.

When a finding sentence is input from the report input screen 233 and the registration button 233e is pressed, the controller 21 transmits an interpretation report (including patient information (patient IDs and the like), inspection information (inspection IDs and the like), a finding sentence, and a thumbnail image) to the report server 1 through the communication section 24.

In the report server 1, when the interpretation report from the image interpretation terminal 2 is received by the communication section 12 (step S4; YES), the controller 11 stores the received interpretation report in the report DB 131 (step S5).

In step S5, the controller 11 issues identification information (report IDs and the like) of the received interpretation reports, and stores the interpretation reports in the report storage area of the report DB 131 in association with the identification information. Furthermore, the controller 11 stores the issued identification information in a record corresponding to the interpretation report in the report management table of the report DB 131. The record corresponding to the interpretation report is a report whose patient information and inspection information match those of the interpretation report.

Next, the controller 11 causes the determination section 14 to execute determining processing of performing natural language analysis on the finding text of the received interpretation report to determine information relating to important findings (step S6).

In step S6, the determination section 14 first acquires a finding sentence that is unstructured data of the interpretation report, and structures each sentence included in the finding sentence by natural language analysis to generate structured data.

For example, the determination section 14 performs morphological analysis on each finding sentence to identify a word in the finding sentence and a part of speech of the word. Next, the determination section 14 performs syntactic analysis on the basis of the morphological analysis result and divides the text into segments.

Next, the determination section 14 identifies a clause related to the determination of the presence or absence of an important finding, such as a clause including a specific part of speech, a clause including a negative expression, or a clause ending with a specific noun, and determines the affirmative sentence/negative sentence of the clause. Examples of the specific part of speech include, for example, “auxiliary verb (.,. , ~)” and “verb (accept, suspect, present)”. Examples of the negative expression include, for example, “. (+auxiliary verb” no”), none” and the like. An example of the “specific noun” in the “phrase ends with the specific noun” includes, for example, “appear and disappear”.

Next, the determination section 14 generates structured data by associating the medical information (site, finding, and disease name) before the identified segment with the determination result of the affirmative sentence and negative sentence. Note that the determination section 14 determines the medical information according to whether the medical information matches a disease name, a finding, or an anatomic name stored in the medical information DB 132. When there is no “specified clause”, it is determined to be a affirmative sentence. For example, a case in which only a disease name or a finding is described is determined to be a affirmative sentence. If a sentence follows the “specified clause”, the same determination is made for the following sentence.

The determination section 14 then acquires the medical information (finding or disease name) of the sentence determined to be the affirmative sentence of the generated structured data. If the acquired medical information (first medical information) matches a phrase corresponding to an important finding, that is, important disease name information or important finding information stored in the important information DB 133, the determination section 14 determines that the sentence includes an important finding. When the medical information of the sentence determined to be a affirmative sentence does not match a phrase corresponding to an important finding or is a negative sentence, the determination section 14 determines that the sentence does not have an important finding.

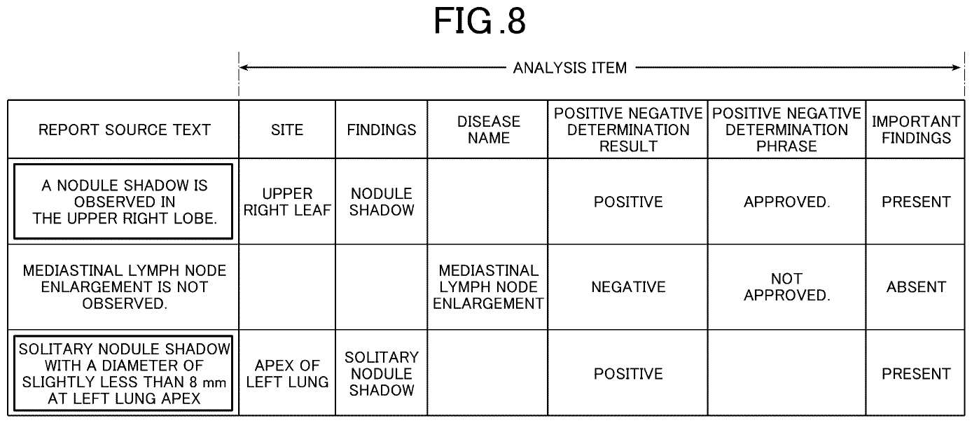

FIG. 8 shows an example of the determination in step S6.

Note that in order to structure modification relations and the like between clauses more accurately, it is also assumed to introduce a semantic analysis method and a contextual analysis method of analyzing relations between a plurality of sentences in a finding sentence.

Furthermore, if it is determined that there is an important finding, the determination section 14 determines the type (urgent/normal), the level (A, B, C, or the like), and the medical care department, which are stored in the important finding DB 133 in association with the word or phrase corresponding to the important finding included in the determined sentence, as the type of important finding, the level of important finding, and the medical care department, respectively.

As described above, the determination section 14 performs natural language analysis on the received interpretation report to determine information relating to important findings. That is, the determination section 14 automatically determines information relating to important findings without depending on the operation of the radiologist. Therefore, it is possible to accurately determine information relating to an important finding in an interpretation report, for example, the presence/absence, type, level, and the like of the important finding, without depending on the radiologist.

In the case where the presence or absence of an important finding is determined based on the presence or absence of a character string that matches a character string registered in advance as a character string corresponding to an important finding, for example, the character string corresponding to the important finding is displayed as “Do not accept--- Fig. However, it is determined that there is an important finding even if a negation such as “yes” continues. Therefore, a large number of character strings that are not important findings are extracted as important findings, and a confirming doctor is forced to perform unnecessary confirmation, with the result that important findings may be overlooked. On the other hand, in the present embodiment, the received interpretation report is subjected to natural language analysis to determine information relating to important findings, and therefore, a message such as “Do not admit--- Fig. For example, in a case where a negation phrase such as continues, it can be determined that it is not an important finding. Therefore, it is possible to prevent a large number of character strings that are not important findings from being extracted as important findings, and to prevent important findings from being overlooked.

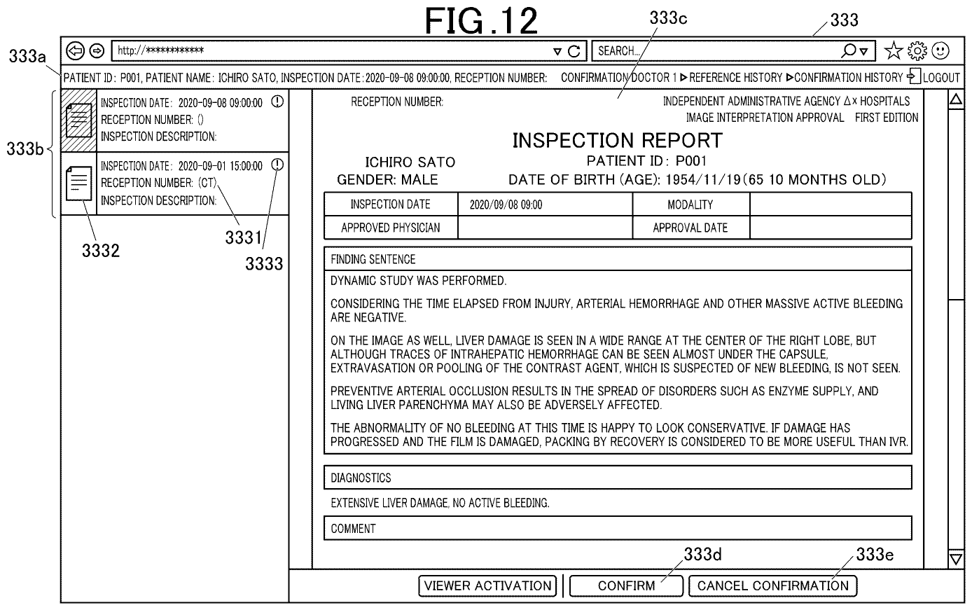

Note that the determination section 14 may determine the importance of the interpretation report on the basis of the determination result of the information relating to the important finding described above. For example, the determination section 14 determines that the interpretation report is important when there is a sentence determined to have an important finding as a result of the determination of the information relating to the important finding in the interpretation report and determines that the interpretation report is not important when there is no sentence determined to have an important finding. Alternatively, the determination section 14 may determine the importance of the interpretation report by determining the level of importance degree of the interpretation report based on the level (level of importance degree) of each sentence obtained by determining the information relating to the important finding in the interpretation report. For example, the determination section 14 may determine, as the level of importance degree of the interpretation report, the highest level among the levels (levels of importance degree) of the respective sentences obtained by the determination of the information relating to important findings in the interpretation report. The determination result of the importance of the interpretation report may be saved in the report DB 131, and output to a report reference screen 333 (FIG. 12) to be described later, similarly to the determination result of the information relating to the important finding.

Next, the controller 11 determines, as a result of the determining processing in step S6, whether or not it has been determined that there is an important finding in the interpretation report (step S7).

If it is determined that there is an important finding in the interpretation report (step S7; YES), the controller 11 generates screen information on a first confirmation screen 234 and outputs (transmits) it to the image interpretation terminal 2 by the communication section 12 (step S8).

Upon receiving the screen information of the first confirmation screen 234 from the report server 1, the interpretation terminal 2 allows the display part 23 to display the first confirmation screen 234.

The first confirmation screen 234 is a screen for the user to confirm whether or not to output the determination result of the information relating to the important finding.

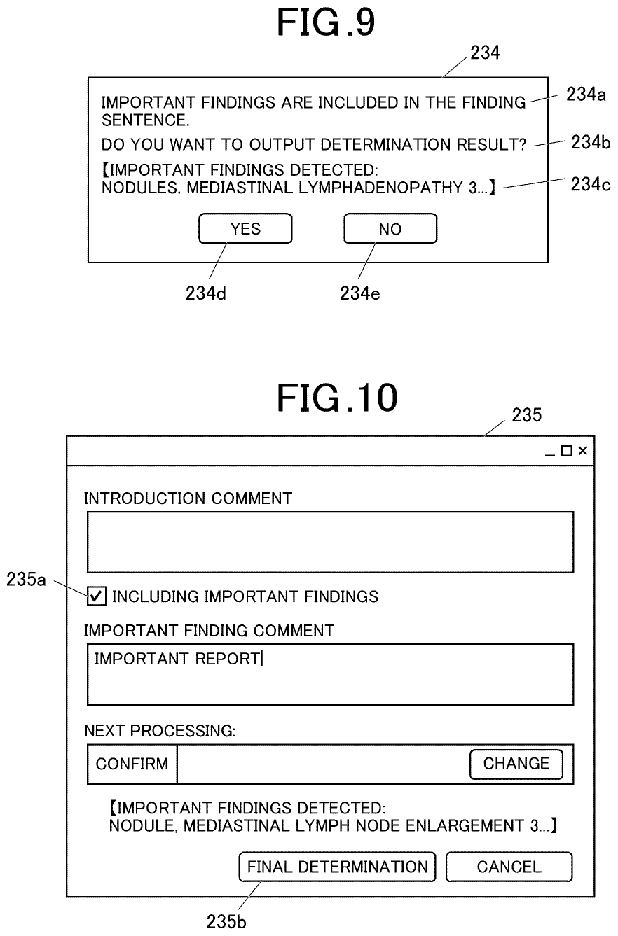

FIG. 9 is a view illustrating an example of the first confirmation screen 234. As illustrated in FIG. 9, the first confirmation screen 234 is provided with a determination result 234a on the presence or absence of information relating to important finding, a confirmation message 234b, a detected important finding 234c, a “YES” button 234d, and a “NO” button 234e. The confirmation message 234b is a message for confirming whether to output the determination result of the information relating to the important finding. The “YES” button 234 is a button for issuing an instruction to output a determination result of information relating to an important finding. Pressing the “YES” button 234d instructs the report server 1 to output the determination result of information relating to important findings. The “no” button 234e is a button for giving an instruction not to output the determination result of the information relating to the important finding. When the “No” button 234e is pressed, the report server 1 is instructed not to output the determination result of the information relating to the important finding. In the present embodiment, when the output of the determination result of the information relating to the important finding is instructed, the determination result is registered in the report management table of the report DB 131, and the determination result is output to the report reference screen 333 (see FIG. 12).

Note that an instruction to only confirm or confirm that there is an important finding is also included in the instruction to output the determination result of the information relating to the important finding. For example, in this case, no confirmation as to whether to output the finding is displayed on the confirmation message 234b, and only a display for confirming whether there is an important finding can be provided.

As described above, the first confirmation screen 234 for confirming whether or not to output the determination result of the information relating to the important finding is displayed on the interpretation terminal 2. Therefore, the radiologist can check the determination result of the information relating to the important finding by the determination section 14, and when there is an error in the determination result, can prevent the determination result from being output.

In a case where the output of the determination result of the information relating to the important findings is instructed in the first confirmation screen 234 (step S9; YES), the controller 11 generates screen information of a second confirmation screen 235 in which “include important findings” is checked, outputs (transmits) the screen information to the image interpretation terminal 2 by the communication section 12 (step S10), and shifts the processing to step S11.

Upon receiving the screen information of the second confirmation screen 235 from the report server 1, the interpretation terminal 2 allows the display part 23 to display the second confirmation screen 235.

The second confirmation screen 235 is a screen having a field for the user to input the determination result of the information relating to the important finding.

FIG. 10 is a view illustrating an example of a second confirmation screen 235 output in step S10. As illustrated in FIG. 10, the second confirmation screen 235 output in step S10 is provided with a checkbox 235a for checking whether an important finding is included, and a final-determination button 235b. In step S10, the second confirmation screen 235 in which the checkbox 235a have been pre-checked is output. The user can input information relating to an important finding (the presence or absence of an important finding) by operating the operation part 22 to unmark or mark. When the checkbox 235a is not particularly operated, the state of the checkbox 235a is regarded as an input by the user. When the final determination button 235b is pressed, the information input on the second confirmation screen 235 is transmitted from the interpretation terminal 2 to the report server 1.

In step S11, the controller 11 determines whether the information relating to the important finding input from the second confirmation screen 235 is different from the determination result of the determination section 14 (step S11).

In step S11, in a case where it is determined that the information relating to the important finding input from the second confirmation screen 235 is the same as the determination result in the determination section 14 (step S11; NO), the controller 11 registers the determination result in the determination section 14 in the report management table of the report DB 131 (step S12), and ends the interpretation report creation processing.

For example, the controller 11 updates the report management table of the report DB 131 by storing, in the record corresponding to the inspection ID of the report creation target, the important finding flag ON, the type of important finding, the level of important finding, the department, and the like as the determination result of the information relating to important finding.

In step S11 If it is determined that the information relating to the important finding input from the second confirmation screen 235 is different from the determination result in the determination section 14 (YES in step S11), the controller 11 changes the determination result in the determination section 14 based on the input information relating to the important finding, registers the important finding flag OFF, which is the changed determination result (second determination result), in the report management table of the report DB 131 (step S13), and ends the interpretation report creation processing.

That is, the controller 11 stores the important finding flag OFF as the determination result of the information relating to the important finding in the record corresponding to the inspection ID of the report creation target in the report management table of the report DB 131.

In this way, the controller 11 displays the second confirmation screen 235 for the user to input information relating to an important finding (presence or absence of an important finding). When the input information is different from the determination result by the determination section 14, the controller 11 changes the determination result of the determination section 14 and registers it in the report management table of the report DB 131. Therefore, for example, when the determination section 14 determines that there is an important finding in an interpretation report that does not include an important finding, the determination result can be changed to “there is no important finding” and registered in the report DB 131.

On the other hand, in step S9, when it is instructed not to output the determination result of the information relating to the important finding (step S9; NO), the controller 11 ends the interpretation report creation processing.

In this way, when it is instructed not to output the determination result of the information relating to the important finding on the first confirmation screen 234, the controller 11 does not output the determination result. Therefore, it is possible not to output the determination result that the determination section 14 erroneously determines that there is an important finding. Thus, for example, the important finding mark 3333 (see FIG. 12) can be prevented from being displayed on the report reference screen 333.

Note that in a case where it is instructed not to output the determination result of the information relating to the important finding in step S9, it indicates that it is instructed not to output the determination result indicating the presence of the important finding. That is, in this case, it can be considered that the user has determined that there is no important finding. Therefore, when it is instructed not to output the determination result of the information relating to the important finding, the controller 11 may register the important finding flag OFF in the record corresponding to the inspection IDs of the report creation targets in the report management table of the report DB 131.

On the other hand, as a result of the determining processing in the determination section 14, if it is determined that there is no important finding in the interpretation report (step S7; NO), the controller 11 generates screen information of the second confirmation screen 235 in which “include important finding” is not checked. The controller 11 outputs (transmits) the screen information to the interpretation terminal 2 by the communication section 12 (step S14). The controller 11 shifts the processing to step S15.

Upon receiving the screen information of the second confirmation screen 235 from the report server 1, the interpretation terminal 2 allows the display part 23 to display the second confirmation screen 235.

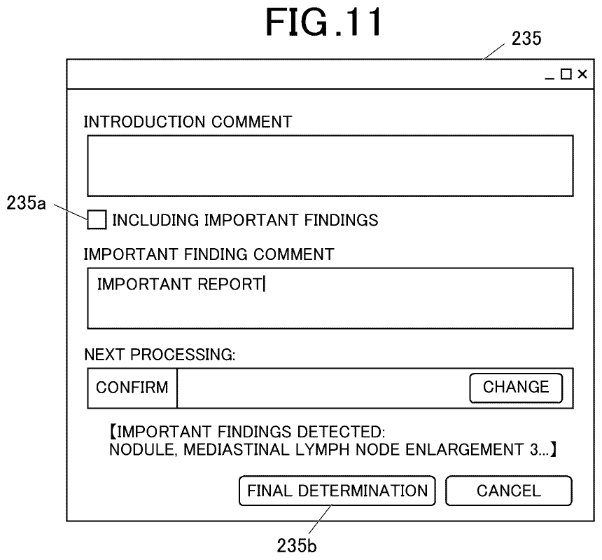

FIG. 11 is a view illustrating an example of a second confirmation screen 235 output in step S14. As illustrated in FIG. 11, the second confirmation screen 235 in which the checkbox 235a is not checked is output as the second confirmation screen 235 output in step S14. The user can input information relating to an important finding (the presence or absence of an important finding) by checking or unchecking the information by operating the operation part 22. When the checkbox 235a is not particularly operated, the state of the checkbox 235a is regarded as an input by the user. When the final determination button 235b is pressed, the information input on the second confirmation screen 235 is transmitted from the interpretation terminal 2 to the report server 1.

In step S15, the controller 11 determines whether the information relating to the important finding input from the second confirmation screen 235 is different from the determination result in the determination section 14 (step S815).

In a case where it is determined that the information relating to the important finding input from the second confirmation screen 235 is the same as the determination result in the determination section 14 (step S15; NO), the controller 11 registers the determination result in the determination section 14 in the report management table (step S16). Then, the controller 11 ends the interpretation report creation processing.

For example, the controller 11 updates the report management table of the report DB 131 by storing the important finding flag OFF as the determination result of the information relating to the important finding in the record corresponding to the inspection of the report creation target.

When determining that the information relating to the important finding input from the second confirmation screen 235 is different from the determination result by the determination section 14 (step S15; YES), the controller 11 changes the determination result by the determination section 14 on the basis of the input information relating to the important finding. The controller 11 registers the changed determination result (second determination result) in the report management table of the report DB 131 (step S17). Then, the controller 11 ends the interpretation report creation processing.

That is, the controller 11 updates the report management table of the report DB 131 by storing the important finding flag ON or the like as the determination result of the information relating to the important finding in the record corresponding to the inspection of the report creation target.

In this manner, the controller 11 displays the second confirmation screen 235 for the user to input information relating to an important finding (presence or absence of an important finding). When the input information is different from the determination result by the determination section 14, the controller 11 changes the determination result of the determination section 14 and registers it in the report management table of the report DB 131. Therefore, for example, when the determination section 14 determines that there is no important finding for the interpretation report including an important finding, the determination result can be changed to “there is an important finding” and registered in the report DB 131. Therefore, in the report reference screen 333 (see FIG. 12), information indicating that an important finding is included can be output in association with the interpretation report.

When the registration of the determination result of the information relating to the important finding in the report DB 131 is completed, the controller 11 outputs the determination result of the information relating to the important finding in response to the access from the reference terminal 3.

For example, when accessed by the reference terminal 3, the controller 11 generates screen information for a report list screen 332 (see FIG. 6) on the basis of the inspection information and the patient information on the inspection for which the report has been created, which are stored in the report management table of the report DB 131. The controller 11 outputs (transmits) the generated screen information to the reference terminal 3 by the communication section 12.

The controller 31 of the reference terminal 3 displays the report list screen 332 on the display part 33 based on the screen information received from the report server 1.

When the inspection information and the patient information of the inspection to be referred to are selected from the inspection information and the patient information displayed on the report list screen 332, the controller 31 of the reference terminal 3 transmits the inspection ID and the like of the selected inspection to the report server 1 by the communication section 34.

When receiving the inspection ID and the like of the selected inspection from the reference terminal 3, the controller 11 generates screen information of the report reference screen 333 of the selected inspection and outputs the screen information to the reference terminal 3 by the communication section 12. The report reference screen 333 is a screen for the user to refer to the interpretation report and the determination result of the important finding.

For example, the controller 11 reads, from the report DB 131, the interpretation reports and the determination result of the report IDs corresponding to the inspection IDs of the selected inspections, and generates the screen information of the report reference screen 333.

The reference terminal 3 displays the report reference screen 333 on the display part 33 based on the screen information received from the report server 1.

FIG. 12 is a view illustrating an example of the report reference screen 333.

As illustrated in FIG. 12, the report reference screen 333 is provided with an information display field 333a, a report list display field 333b, an interpretation report display field 333c, a confirmation button 333d, a confirmation cancel button 333e, and the like.

The information display field 333a displays inspection information and patient information of the target examination.

The report list display field 333b displays a list of interpretation reports generated in the past for the patient of the target examination. For example, information (inspection date, reception number, inspection description, and the like) 3331 for specifying the inspection of each interpretation report and a display 3332 indicating read/unread are displayed in a list. The indication 3332 indicating read/unread is displayed in such a manner that, for example, read and unread are distinguished by different colors. Read/unread may be displayed by characters. In the report list display field 333b, an important finding mark 3333, which is information indicating that an important finding is included in the row of the interpretation report determined to include an important finding, is displayed. The interpretation report determined to have an important finding is an interpretation report with the important finding flag being ON.

The interpretation report display field 333c displays an interpretation report of the selected inspection.

The confirmation button 333d is a button for registering that the interpretation report displayed in the interpretation report display field 333c has been confirmed. When the confirm button 333d is pressed by the operation part 32, the controller 31 transmits a confirmation completion notification of the interpretation report displayed in the interpretation report display field 333c to the report server 1 by the communication section 34 in association with the inspection information (inspection ID and the like) corresponding to the interpretation report. Upon receiving the confirmation completion notification of the interpretation report, the controller 31 of the report server 1 registers a confirmation completion flag indicating that the report has been confirmed (read) in the record of the corresponding inspection ID in the report management table of the report DB 131.

As illustrated in FIG. 12, in the report reference screen 333, an important finding mark 3333 is displayed in association with the interpretation report in which the determination result of the information relating to the important finding is determined as the presence of the important finding. The important finding mark 3333 is information indicating that an important finding is included, which is a determination result of information relating to an important finding. Therefore, the confirming doctor who confirms the interpretation report can recognize the interpretation report including the important finding, and can prevent the confirmation of the important finding from being omitted.

Note that FIG. 12 illustrates the case of outputting (displaying) an important finding mark 3333 as the determination result of the information relating to the important finding on the list of the interpretation report. However, the controller 11 may output (display) the important finding mark 3333 as the determination result of the information relating to the important finding on the interpretation report. In the present embodiment, the determination result is not displayed for the interpretation report in which the important finding flag is OFF. However, the controller 11 may output (display) a mark indicating that the finding is not an important finding on the list or the interpretation report. In addition, the controller 11 may output (display) the phrase determined as the important finding included in the interpretation report and the type or level of the important finding together with the important finding mark 3333 or instead of the important finding mark 3333.

As described above, according to the controller 11 of the report server 1, the determination section 14 that performs the natural language analysis on the interpretation report and determines the information relating to the important finding performs the determination. Then, the determination result by the determination section 14 is output.

Therefore, it is possible to accurately determine information relating to an important finding in an interpretation report and output the determination result without depending on the skill of a radiologist. Therefore, it is possible to prevent the checker of the interpretation report from missing an important finding.

Note that the description in the above embodiment is a preferred example of the analysis device, the program, the analysis system, and the analysis method according to the present invention, and the present invention is not limited to this.

For example, when the determination section 14 determines that there is an important finding, the controller 11 of the report server 1 may output the determination result without outputting the first confirmation screen 234 for confirming whether to output the determination result.

For example, in the interpretation report creation processing, when it is determined in step S7 that there is an important finding, the controller 11 may proceed to step S12. That is, when it is determined that there is an important finding, the controller 11 may store and update the determination result (important finding flag ON) of the information relating to the important finding in the record corresponding to the inspection ID of the target inspection in the report management table of the report DB 131. Then, when the output of the report reference screen 333 of the target examination is instructed from the reference terminal 3, the controller 11 may output the report reference screen in which the important finding mark 3333 is displayed on the row of the target examination or the interpretation report of the report list display field 333b in the report reference screen 333.

Alternatively, if it is determined in step S7 that there is an important finding, the controller 11 may proceed to step S10 and output the second confirmation screen 235. The controller 11 may store and update the important finding flag ON or OFF as the determination result of the information relating to the important finding in the record corresponding to the inspection ID of the target inspection of the report management table of the report DB 131 according to the input of the user in the second confirmation screen 235.

Furthermore, when it is instructed to output the determination result on the first confirmation screen 234, the controller 11 may output the determination result without outputting the second confirmation screen 235. For example, in the interpretation report creation processing, when it is determined in step S9 that the determination result is to be outputted, the controller 11 may proceed to step S12. That is, when the controller 11 determines that the determination result is to be outputted in step S9, the controller 11 may store the important finding flag ON as the determination result of the information relating to the important finding in the record corresponding to the inspection IDs of the target inspections in the report management table of the report DB 131 to update the record. When the output of the report reference screen 333 of the target examination is instructed from the reference terminal 3, the controller 11 may output the report reference screen in which the important finding mark 3333 is displayed on the row of the target examination or the interpretation report of the report list display field 333b of the report reference screen 333.

Furthermore, when it is instructed not to output the determination result on the first confirmation screen 234, the controller 11 may output the second confirmation screen 235 to confirm with the user that information relating to important findings is not included.

In the interpretation report creation processing, even when it is determined that there is no important finding in step S7, the controller 11 may output the first confirmation screen for confirming whether or not to output the determination result of the information relating to the important finding. An example of the first confirmation screen in this case includes, for example, a message such as “No important finding is included in the finding sentence. Do you want to register?”, and a “Yes” button, a “No” button, and the like. If the “Yes” button is pressed in the first confirmation screen in this case, the controller 11 moves the process to step S14. Alternatively, the controller 11 may omit display of the second confirmation screen 235 and register the important finding flag being OFF in the report management table of the report DB 131. When the “NO” button is pressed, the controller 11 may shift the process to step S10.

Further, the determination section 14 may perform the natural language analysis and the determination of the information relating to the important finding using a machine learning model. The machine learning model in this case is a machine learning model obtained by machine learning using, as labeled data, a data set of an interpretation report and information relating to important findings.

For example, the interpretation report in which each sentence of the finding sentences of the interpretation report is divided into clauses and the clauses including the specific part of speech, the negative expression, and the specific noun described in step S6 of FIG. 5 are tagged with “the specific part of speech”, “the negative expression”, and “the specific noun” is set as the interpretation report of the labeled data. Information (the presence or absence of an important finding, the important finding (disease name, finding), the site, type, level, department, and the like of the important finding) about the important finding corresponding to each finding sentence of the interpretation report is set as information relating to the important finding of the labeled data. The determination section 14 is configured to have a machine learning model obtained by machine learning of a data set of the interpretation report and the information relating to important findings. The determination section 14 performs natural language analysis on the interpretation report received from the interpretation terminal 2 by using the machine learning model, thereby determining information relating to the important finding in the received interpretation report.

Further, the controller 11 may function as the weighting section to weight the information relating to the important finding and output the determination result of the information relating to the important finding in accordance with the weighting.

As an example of weighting, for example, the controller 11 calculates, as a weight, the sum of the numbers (the number of appearances) of important findings included in the finding sentence. For example, when the finding sentence includes three “lung cancer” and one “nodule” that are important findings, the weight is 4.

Alternatively, the controller 11 may calculate, as the weight of an important finding, the number of important findings (the number of appearances) included in a finding sentence. For example, when a finding sentence includes three “lung cancer” and one “nodule” that are important findings, the weight of the “lung cancer” is 3 and the weight of the “nodule” is 1.

Note that the number of appearances may be directly used as a weight, or the number of appearances may be divided into sections and ranked for weighting.

Further, the controller 11 may perform weighting based on whether or not the important finding included in the finding sentence is a finding of a site different from the inspection target site. For example, the controller 11 may increase the weighting in a case where the site of the inspection purpose and the site where the important finding exists are the same. This can contribute to prevention of oversight of an important finding for the inspection purpose. Furthermore, the controller 11 may increase the weighting in a case where the part targeted for the inspection is different from the part in which the important finding exists. This can contribute to prevention of oversight of a finding found for a purpose other than the inspection.

As an output example of the determination result of the information relating to the important finding according to the weighting, for example, the controller 11 may display the important finding mark 3333 displayed on the report reference screen 333 in a color corresponding to the calculated weight. For example, the controller 11 may display in light red when the weight is low, and display in deeper red as the weight increases. When the weight is calculated as the sum of the number of important findings (the number of appearances) included in the finding sentence, the controller 11 may display the number as it is in the important finding mark 3333. In a case where the number (appearance number) for each important finding is calculated as the weight of the important finding, the controller 11 may display the number (appearance number) for each important finding on the report reference screen 333.

Further, the controller 11 may change the output mode of the determination result based on the determination result of the information relating to the important finding. For example, if the site where the important finding detected from the finding sentence exists does not match the inspection site of the target inspection, the controller 11 may display an important finding mark in a form prompting an additional inspection. Alternatively, the controller 11 may display a message prompting an additional inspection.

Furthermore, for example, when the medical care department handling the important finding detected from the finding sentence does not match the medical care department included in the inspection information on the target examination, the controller 11 may display an important finding mark in a form indicating that it is better to have a doctor in another medical care department check the important finding. Alternatively, the controller 11 may display a message notifying that it is better to ask a doctor in another medical care department for confirmation.

When the type of the important finding is urgent, the controller 11 may display an important finding mark indicating urgent. Alternatively, the controller 11 may display a message indicating urgency.

Furthermore, although the controller 11 acquires, from the RIS, the information on the inspection that is the target of report creation and is to be registered in the report management table of the report DB 131 in the embodiment described above, the information may be acquired from the PACS if there is no RIS.

Furthermore, although the controller 11 activates (displays) the report input screen 233 for creating an interpretation report on the inspection when the inspection is selected from the report list screen 232 in the embodiment described above, the method of activating the report input screen 233 is not limited thereto. For example, the controller 11 may activate the report input screen 233 in response to pressing of an inspection list of the PACS or a button from the viewer. In that case, the controller 11 acquires inspection information such as an examination ID from the calling source (PACS) and activates the report input screen 233.

The above embodiment has been described with the example in which the report server 1 has the functions as the analysis device of the present invention, that is, the functions as the determination section, the output section, the generation section, the acquisition section, the storage, and the weighting section. Alternatively, these function may be provided in the image interpretation terminal 2.

For example, the controller 21 of the interpretation terminal 2 may execute each step of the interpretation report creation processing shown in FIG. 5 in cooperation with a program. In this case, the controller 21 acquires the inspection information from the report server 1, generates the report list screen 232, and causes the display part 23 to display it. When the inspection for which the interpretation report is to be created is selected, the controller 21 allows the display part 23 to display a report input screen 233 on the basis of the inspection information and the patient information on the selected inspection. Next, the controller 21 sends the input interpretation report together with the inspection IDs to the report server 1 for storage in the report DB 131. The interpretation terminal 2 is configured to include the determination section, and the determination section performs determining processing of the interpretation report. Next, the controller 21 performs processing in step S7 to S17 in accordance with the determination result of the determination section and outputs the determination result to the report server 1. the report server 1 stores the determination result in the report DB 131, and outputs a report reference screen including the determination result to the reference terminal 3 in response to access from the reference terminal 3.

The functions of the determination section, the output section, the generation section, the acquisition section, the storage, and the weighting section according to the present invention may be distributed among a plurality of devices such as the report server 1 and the image interpretation terminal 2.

Furthermore, although an example in which a ROM or an HDD is used as a computer-readable medium storing a program for executing each processing has been disclosed in the above description, it is not limited to this example. As other computer-readable media, a nonvolatile memory such as a flash memory and a portable recording medium such as a CD-ROM are also applicable. Further, a carrier wave may be applied as a medium for providing program data via a communication line.

In addition, the detailed configuration and detailed operation of each unit constituting the analysis device or the analysis system can be appropriately changed without departing from the scope of the present invention.

### Industrial Applicability

The present invention can be used in the medical field.

### REFERENCE SIGNS LIST

100 report system
1 report server
11 controller
12 communication section
13 storage
131 report DB
132 medical information DB
133 important findings DB
14 determination section
15 bus
2 interpretation terminal
3 reference terminal
21, 31 controller
22, 32 operation part
23, 33 display part
24, 34 communication section
25, 35 storage
231 dictionary management screen
232, 332 report list screen
233 report input screen
234 first confirmation screen
235 second confirmation screen
333 report reference screen
N Communication network

1. An analysis device comprising:
a hardware processor that performs natural language analysis on an interpretation report to determine information relating to an important finding; and
outputs a determination result.

2. The analysis device according to claim 1, wherein the information relating to the important finding is information on presence or absence of the important finding.

3. The analysis device according to claim 1, wherein the information relating to the important finding is information on a type or a level of the important finding.

4. The analysis device according to claim 1, wherein the hardware processor outputs a first confirmation screen for confirming whether or not to output the determination result based on the information relating to the important finding before outputting the determination result.

5. The analysis device according to claim 4, wherein in response to an instruction to output the determination result in the first confirmation screen,
the hardware processor outputs the determination result.

6. The analysis device according to claim 4, wherein in response to an instruction not to output the determination result in the first confirmation screen,
the hardware processor does not output the determination result.

7. The analysis device according to claim 1, wherein the hardware processor outputs, in association with the interpretation report, information indicating that the important finding is included in the interpretation report, based on the determination result.

8. The analysis device according to claim 1, wherein the natural language analysis is analysis using a machine learning model.

9. The analysis device according to claim 8, wherein the machine learning model is a learning model that has learned a data set of an interpretation report and information relating to an important finding.

10. The analysis device according to claim 1, comprising:
a storage that stores, as important finding information, disease name information that is important or finding information that is important among disease name information or finding information, wherein
the hardware processor
structures unstructured data acquired from the interpretation report by the natural language analysis to generate structured data;
acquires first medical information from the structured data; and
determines, based on the important finding information, whether or not the first medical information is an important finding.

11. The analysis device according to claim 4, wherein the hardware processor outputs a second confirmation screen on which a user inputs information relating to an important finding, and
the hardware processor changes the determination result and outputs the changed determination result as a second determination result, based on the information relating to the important finding input by the user.

12. The analysis device according to claim 4, wherein in response to determination, based on the information relating to the important finding determined by the hardware processor, that the interpretation report does not include the important finding, the hardware processor outputs a second confirmation screen on which a user inputs information relating to an important finding, and
the hardware processor changes the determination result and outputs the changed determination result as a second determination result, based on the information relating to the important finding input by the user in the second confirmation screen.

13. The analysis device according to claim 4, wherein in response to an instruction in the first confirmation screen whether or not to output the determination result,
the hardware processor outputs a second confirmation screen based on the instruction before outputting the determination result.

14. The analysis device according to claim 1, wherein the hardware processor performs weighting on the information relating to the important finding, and outputs the determination result in accordance with the weighting.

15. The analysis device according to claim 1, wherein the hardware processor determines an importance of the interpretation report based on the determination result of the information relating to the important finding.

16. The analysis device according to claim 15, wherein the hardware processor determines the importance of the interpretation report by determining whether the interpretation report is important or not or a level of an importance degree of the interpretation report based on the determination result of the information relating to the important finding.

17. The analysis device according to claim 1, wherein the natural language analysis includes at least one of syntactic analysis, semantic analysis, and contextual analysis.

18. A non-transitory computer-readable recording medium storing a program for causing a computer to achieve:
determining of performing natural language analysis on an interpretation report and determining information relating to an important finding; and
outputting of outputting a determination result by the determining.

19. The recording medium according to claim 18, wherein the information relating to the important finding is information on presence or absence of the important finding.

20. The recording medium according to claim 18, wherein the information relating to the important finding is information on a type or a level of the important finding.

21. The recording medium according to claim 18, wherein the outputting outputs a first confirmation screen for confirming whether or not to output the determination result based on the information relating to the important finding before outputting the determination result.

22. The recording medium according to claim 21, wherein in response to an instruction to output the determination result in the first confirmation screen,
the outputting outputs the determination result.

23. The recording medium according to claim 21, wherein in response to an instruction not to output the determination result in the first confirmation screen,
the outputting does not output the determination result.

24. The recording medium according to claim 18, wherein the outputting outputs, in association with the interpretation report, information indicating that the important finding is included in the interpretation report, based on the determination result.

25. The recording medium according to claim 18, wherein the natural language analysis is analysis using a machine learning model.

26. The recording medium according to claim 25, wherein the machine learning model is a learning model that has learned a data set of an interpretation report and information relating to an important finding.

27. The recording medium according to claim 18, wherein the program causes the computer to perform:
generating of structuring unstructured data acquired from the interpretation report by the natural language analysis to generate structured data;
acquiring of acquiring first medical information from the structured data; and
storing of storing, as important finding information, disease name information that is important or finding information that is important among disease name information or finding information, and
the determining determines, based on the important finding information, whether or not the first medical information is an important finding.

28. The recording medium according to claim 21, wherein the outputting outputs a second confirmation screen on which a user inputs information relating to an important finding, and
the outputting changes the determination result and outputs the changed determination result as a second determination result, based on the information relating to the important finding input by the user.

29. The recording medium according to claim 21, wherein in response to determination, based on the information relating to the important finding determined by the determining, that the interpretation report does not include the important finding, the outputting outputs a second confirmation screen on which a user inputs information relating to an important finding, and
the outputting changes the determination result and outputs the changed determination result as a second determination result, based on the information relating to the important finding input by the user in the second confirmation screen.

30. The recording medium according to claim 21, wherein in response to an instruction in the first confirmation screen whether or not to output the determination result,
the outputting outputs a second confirmation screen based on the instruction before outputting the determination result.

31. The recording medium according to claim 18, wherein the program causes the computer to perform weighting of performing weighting on the information relating to the important finding, and
the outputting outputs the determination result in accordance with the weighting.

32. The recording medium according to claim 18, wherein the determining determines an importance of the interpretation report based on the determination result of the information relating to the important finding.

33. The recording medium according to claim 32, wherein the determining determines the importance of the interpretation report by determining whether the interpretation report is important or not or a level of an importance degree of the interpretation report based on the determination result of the information relating to the important finding.

34. The recording medium according to claim 18, wherein the natural language analysis includes at least one of syntactic analysis, semantic analysis, and contextual analysis.

35. An analysis system comprising:
a hardware processor that performs natural language analysis on an interpretation report to determine information relating to an important finding; and
outputs a determination result.

36. An analysis method comprising:
determining of performing natural language analysis on an interpretation report to determine information relating to an important finding; and
outputting of outputting a determination result in the determining.
